(19) United States
(12) Patent Application Publication
(10) Pub. No.: US 2013/0068690 A1
(43) Pub. Date: Mar. 21, 2013

(54) ADSORPTION DEVICES, SYSTEMS AND METHODS

(71) Applicants: THE UNIVERSITY OF NORTH CAROLINA AT CH, Chapel Hill, NC (US); NORTH CAROLINA STATE UNIVERSITY, Raleigh, NC (US)

(72) Inventors: Marian G. McCord, Apex, NC (US); Melanie S. Joy, Chapel HIll, NC (US)

(73) Assignees: NORTH CAROLINA STATE UNIVERSITY, Raleigh, NC (US); THE UNIVERSITY OF NORTH CAROLINA AT CHAPEL HILL, Chapel HIll, NC (US)

(21) Appl. No.: 13/655,095

(22) Filed: Oct. 18, 2012

Related U.S. Application Data
(63) Continuation of application No. PCT/US2011/033252, filed on Apr. 20, 2011.
(60) Provisional application No. 61/326,093, filed on Apr. 20, 2010.

(51) Int. Cl. B01J 20/28; A61M 1/16; C23C 4/10; B01J 20/26; B05D 5/00; B01J 20/22; B01J 20/24; B01D 39/08; C23C 16/50

(52) U.S. Cl. 210/646; 210/502.1; 210/663; 427/180; 427/569; 427/453; 252/184; 210/483

(57) ABSTRACT

Adsorption devices, systems, and methods are provided. In one embodiment, an adsorption device includes a fabric and an adsorbing complex attached at least partially attached to a surface of the fabric, the adsorbing complex being reactive to selectively adsorb a substance from a liquid passing through the fabric to remove at least a portion of the substance from the liquid. In one embodiment, the adsorption device can be incorporated into an extracorporeal blood circuit adapted to filter phosphate from blood of a hemodialysis patient without altering chemistry of the blood.

### CROSS-REFERENCE TO RELATED APPLICATIONS

This application a continuation of PCT/US2011/033252, filed on Apr. 20, 2011, and which claims the benefit of and priority to U.S. Provisional Patent Application Ser. No. 61/326,093, filed Apr. 20, 2010, the entire disclosures of which are incorporated by reference herein.

### GOVERNMENT INTEREST

This invention was made with U.S. Government support under Grant No. UL1RR025747 from the National Center for Research Resources; Grant No. 09-1455 from the University of North Carolina (UNC) National Institute of Health (NIH) Clinical and Translational Science Award (CTSA); Grant No. 50KR41012 from UNC NIH CTSA. Thus, the U.S. Government has certain rights in the invention.

### TECHNICAL FIELD

The subject matter disclosed herein relates to adsorption devices, systems, and methods. More particularly, the subject matter disclosed herein relates to adsorption devices, systems, and methods for selectively and at least partially adsorbing undesired material present for example in a liquid, without altering other chemical concentrations that may be present in the liquid.

### BACKGROUND

In general, undesirable material such as substances may be present in various liquids, including wastewater, natural bodies of water, blood, and bodily fluids. One example of undesirable substances includes phosphate ions present in blood or wastewater. Phosphates from wastewater contribute to a major source of organic pollution in natural bodies of water. Elevated levels of phosphate ions in blood can result in a medical condition known as hyperphosphatemia, which is an electrolyte disturbance in the blood, and is common in dialysis patients. For example, approximately 90% of hemodialysis, or dialysis patients have elevated blood phosphate levels. Hyperphosphatemia is linked to significant morbidity and mortality rates in hemodialysis patients. Currently, diet restrictions and oral phosphate binding medications are prescribed to lower phosphate levels in blood, but are not always effective.

Phosphorus is introduced to patients through the diet and is inherently related to dietary protein. Phosphate levels can be reduced in patients using conventional methods such as adhering to strict diets that restrict phosphate intake and/or using oral phosphate binding medications. Phosphate levels can also be lowered to some degree during hemodialysis. Lowering the phosphate load by decreasing protein intake may be counterproductive however, as protein deficiency may lead to an increased death risk. Thus, a mechanism for controlling blood phosphates while maintaining dietary protein intake is most optimal. Even with conventional therapy such as phosphate binding medications, approximately 50% of hemodialysis patients still may experience hyperphosphatemia. Oral phosphate-binding drugs, e.g., calcium acetate (Phoslo®), sevelamer hydrochloride (RenaGel®) and lanthanum carbonate (Fosrenol®) possess negative characteristics including finite binding capacity (40-50%), poor patient compliance, and undesirable side effects.

Hemodialysis results in some lowering of blood phosphates. Hemodialysis is a technique used in the medical field for treating patients suffering from end stage renal disease (ESRD) and/or other kidney disorders. During hemodialysis, blood may be removed from the body and externally processed through a hemodialysis system. Hemodialysis systems can include a series, or circuit, of one or more devices for extracting blood from a patient's body, feeding the blood through a dialyzer to remove waste products in the blood, and feeding purified blood back into the patient's body. Dialyzers include devices serving as “artificial kidneys” for purifying blood in patients with kidney disorders, and are well known in the art. Phosphorus removal during the dialysis procedure is governed by the passive process of simple diffusion, whereby solute follows a concentration gradient from high to low concentrations. Additionally, current dialysis conditions are not prescribed based on achievement of some level of phosphate removal. During a recommended four-hour hemodialysis session, approximately 150 mg of phosphorus is removed in the first 30 minutes, while only 65 mg is removed during the last 30 minutes for a 4-hour average total of 800 mg per session. The result is a net positive balance of phosphorus of 1128 mg per week and at least 160 mg per day. To prevent this positive balance, it is necessary to increase phosphorus removal during the three-times-a-week hemodialysis session by approximately 376 mg per session.

Accordingly, there is a need for adsorption devices, systems, and methods of controlling blood phosphate levels in hemodialysis patients to reduce the incidence of high phosphate-associated mortality. In one aspect, adsorption devices, systems, and methods that can be incorporated into existing hemodialysis circuits are desirable, as reliance upon patient compliance with dietary restrictions and/or medication dosing can be diminished, out-of-pocket medication costs can be reduced, and medication-related side effects can be avoided.

### SUMMARY

According to one aspect, the subject matter described herein includes an adsorption device. The device can comprise a fabric substrate and an adsorbing complex finish that is chemically grafted or physically adhered to the surface of the fabric substrate. The adsorbing complex may be adapted to adsorb undesirable ions present in a liquid. In one embodiment, undesired concentrations of substances found in bodily fluids, for example, blood can be filtered or adsorbed via adsorptive devices, methods, and systems described. As used herein, the terms “hemoadsorption” and “hemofiltration” are indicative of removing undesired substances from blood via adsorption of the substances onto adsorptive devices and/or systems. The terms “hemoadsorption” and “hemofiltration” are synonymous terms which may be used interchangeably in this application.

As used herein the term “selective” adsorptive compounds can include compounds having a specific chemical structure for specifically targeting, or adsorbing undesired or targeted substances, impurities or particles thereby removing the particles from an aqueous solution. For example, selective adsorptive compounds may comprise a compound having a chemical structure for specifically targeting adsorption of phosphates to remove phosphates from an aqueous solution. Adsorptive devices, methods, and systems disclosed herein may comprise selective and/or non-selective adsorptive compounds which can be chemically grafted thereto for removal of specified or non-specified impurities.

### BRIEF DESCRIPTION OF THE DRAWINGS

Preferred embodiments of the subject matter described herein will now be described with reference to the accompanying drawings, of which:

FIG. 1 is a diagram illustrating possible steps in creating an adsorption device according to an embodiment of the subject matter described herein;

FIG. 2 is a schematic diagram illustrating a processing device used to create an adsorption device according to an embodiment of the subject matter described herein;

FIGS. 3A to 3E are scanning electron microscope (SEM) images of an adsorption device according to an embodiment of the subject matter described herein;

FIG. 4 is a schematic diagram illustrating a processing device used to prepare the surface of an existing fabric for treatment with an adsorption complex according to an embodiment of the subject matter described herein;

FIGS. 5 to 8 are graphical illustrations indicating amounts and/or percentages of phosphate adsorption using an adsorption device according to an embodiment of the subject matter described herein;

FIG. 9 is a schematic diagram illustrating an adsorption method according to an embodiment of the subject matter described herein;

FIG. 10 is a schematic diagram illustrating an in vivo hemodialysis circuitry and placement of adsorption device representing an adsorption system according to an embodiment of the subject matter described herein;

FIG. 11 illustrates an adsorption system according to an embodiment of the subject matter described herein;

FIG. 12 is a schematic diagram of an adsorption system according to an embodiment of the subject matter described herein; and

FIG. 13 is a flow chart illustrating steps of a method for adsorption of undesired substances using adsorption systems and/or devices according to an embodiment of the subject matter described herein.

### DETAILED DESCRIPTION

In accordance with the subject matter disclosed herein, adsorption devices, systems, and methods are provided. Reference will now be made in detail to possible aspects or embodiments of the subject matter herein, one or more examples of which are shown in the figures. Each example is provided to explain the subject matter and not as a limitation. In fact, features illustrated or described as part of one embodiment can be used in another embodiment to yield still a further embodiment. It is intended that the subject matter disclosed and envisioned herein covers such modifications and variations.

As illustrated in the various figures, some sizes of structures or portions are exaggerated relative to other structures or portions for illustrative purposes and, thus, are provided to illustrate the general structures of the present subject matter. Furthermore, relative terms such as “on”, “above”, “upper”, “top”, “lower”, or “bottom” are used herein to describe one structure's or portion's relationship to another structure or portion as illustrated in the figures. It will be understood that relative terms such as “on”, “above”, “upper”, “top”, “lower” or “bottom” are intended to encompass different orientations of the device in addition to the orientation depicted in the figures. For example, if the device in the figures is turned over, structure or portion described as “above” other structures or portions would now be oriented “below” the other structures or portions. Likewise, if devices in the figures are rotated along an axis, structure or portion described as “above”, other structures or portions would now be oriented “next to” or “left of” the other structures or portions. Like numbers refer to like elements throughout.

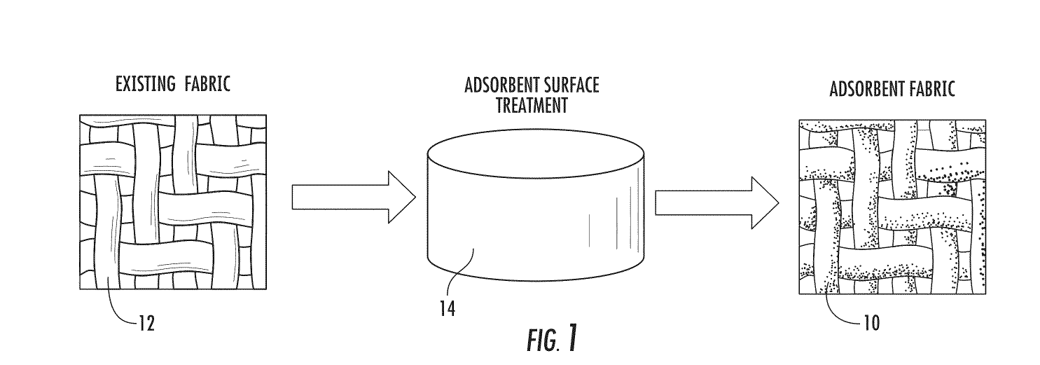

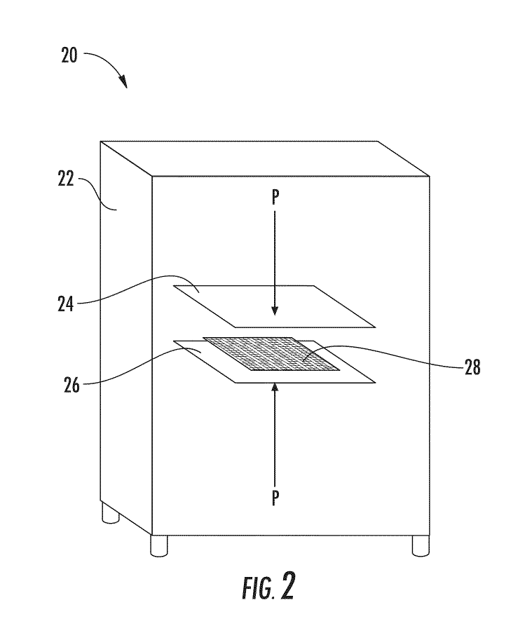

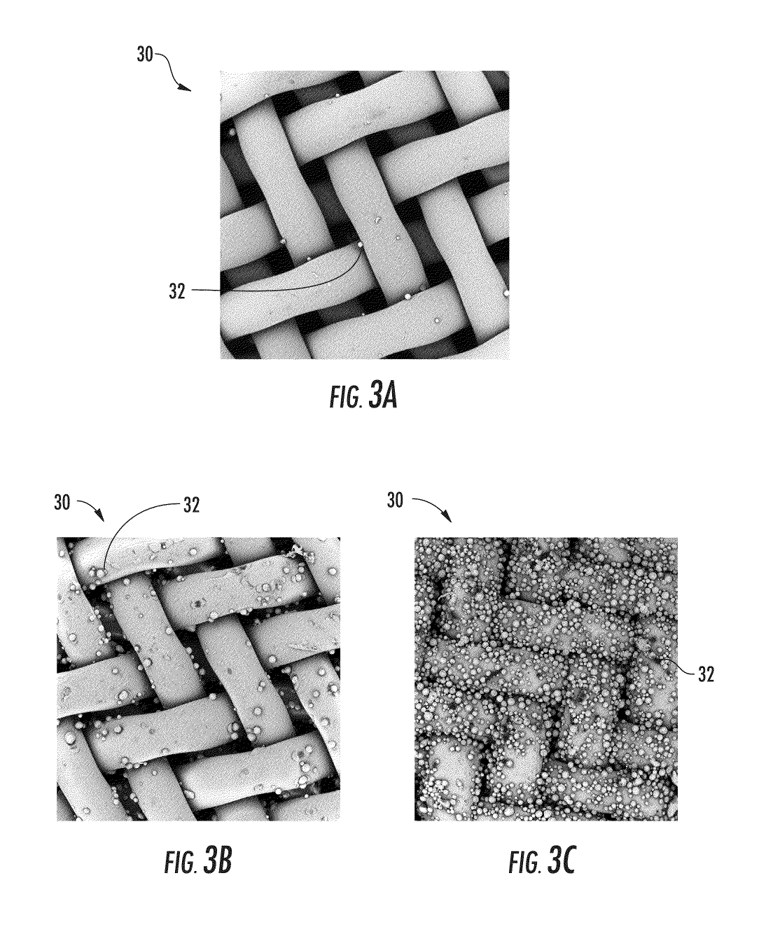

Referring now to FIG. 1, an adsorption device 10 is illustrated. In one embodiment, adsorption device 10 can comprise an adsorbent fabric formed by treating an existing fabric 12 with an adsorbent surface treatment 14. In one embodiment and without limitation, adsorbent surface treatment 14 can comprise binding or chemically grafting existing fabric 12 with trimesic acid (TMA) alone and/or TMA complexed with alumina, alumina alone, (see, FIGS. 2 and 3) or aluminum hydroxide, or aluminum hydroxide precipitates described further herein. In other embodiments, adsorbent surface treatments 14 comprise synthesizing existing fabric 12 with phosphate binding proteins such as peptide sequences with or without surrounding amino acids and binding domains. Such binding proteins can comprise specific phosphate binding domains for selectively adsorbing phosphate. Binding domains are part of the phosphate binding protein. The whole protein, the domain, or both can be bound to existing fabric 12. In addition, the domain plus surrounding amino acids that are part of the whole protein can be bound to existing fabric.

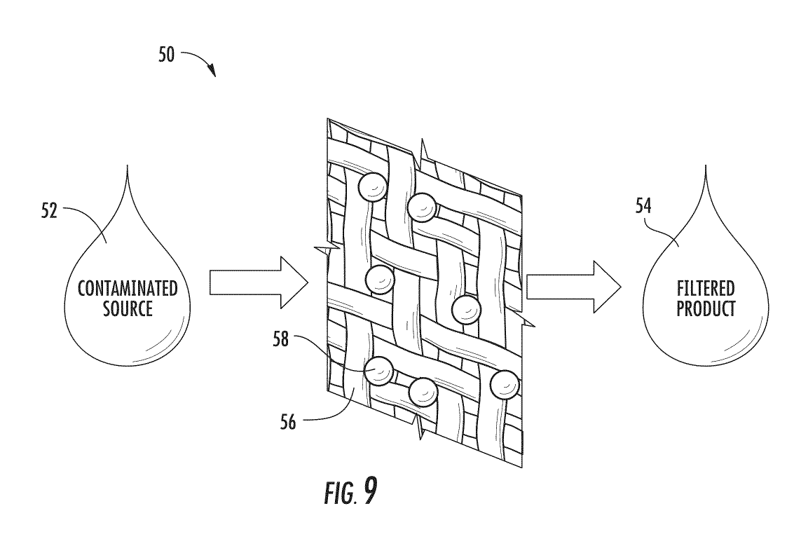

Novel textile finishing processes can be used to bind selective adsorptive compounds onto existing filtration fabrics and/or other filtration platforms to create a safe, effective adsorbent device 10. In one embodiment, adsorptive compounds can include compounds for selectively adsorbing phosphates. For example, adsorption device 10 can comprise an adsorbent hemofiltration fabric for filtering blood during hemodialysis such that phosphates are adsorbed onto the fabric and effectively removed from the blood. In one aspect, adsorption device 10 can be used in filter systems (FIGS. 10-12) to treat hyperphosphatemia in patients. Adsorption device 10 can also comprise a filtration fabric for filtering domestic wastewater to adsorb phosphates, or any undesirable ion or contaminant, thereby removing a major source of organic pollution before it reaches bays, lakes, and/or other bodies of water. Adsorption device 10 can also comprise filtration fabrics or materials for treatment of sepsis/septic shock and acute liver failure by adsorbing harmful particles and/or contaminants onto the fabric to effectively remove the contaminants from the body and/or bodily fluids. Adsorption devices, systems, and methods described herein can be used to treat any contaminated or potentially contaminated source.

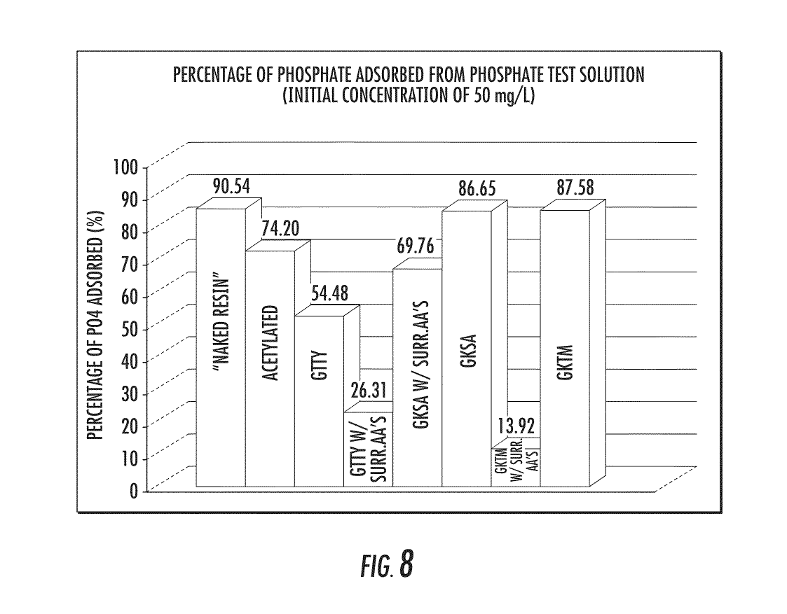

Fabric 12 can comprise any type of fabric, textile material, fabric substrate, or fibrous material or substrate. Fabric 12 can comprise a woven or non-woven fibrous substrate. In one aspect, existing fabric 12 can for example and without limitation comprise a medical grade polyester woven monofilament filter fabric, such as polyethylene terephthalate (PET). In other aspects, existing fabric 12 can comprise polyester fabric, non-polyester fabric, woven polyester monofilament, polymer and filament, or a combination thereof. However, any suitable fabric is contemplated. When existing filter fabric 12 undergoes adsorbent surface treatment 14, it can transform into an adsorbent fabric, or adsorbent device 10 enhanced by a permanently bound phosphate selective adsorptive mechanism or complex. Adsorbent surface treatment 14 can comprise a novel surface treatment for chemically grafting or physically attaching selective adsorptive compounds to existing filtration fabrics, including blood filtration fabrics thereby forming adsorption devices. Chemically grafting an adsorbing complex to a surface of the fabric enables a chemically reactive adsorbing complex to selectively and permanently adsorb any suitable substance, chemical material, or targeted ion from an aqueous solution including but not limited to blood or water. Substances adsorbed by adsorbing complex can for example comprise unwanted chemical materials such as phosphates or toxic metal ions. In one aspect, adsorbent surface treatment 14 can comprise a novel surface treatment to chemically graft the adsorptive compounds to the fabric of an existing blood filter cartridge thereby forming adsorption systems (e.g., FIGS. 9-11). In one embodiment, adsorbent surface treatment 14 can transform existing fabric 12 into an adsorbing device 10 comprising a medical grade filter fabric with a phosphorus-selective adsorbent finish. That is, adsorption device 10 may be used to specifically target the adsorption of phosphate ions, or particles from a source aqueous solution.

Furthermore, this approach can be extended to downstream applications such as endotoxin and host cell DNA removal from biotherapeutics. Polymyxin B (a peptide antibiotic) affinity chromatography is currently used to remove endotoxins following anion exchange chromatography. Although it may be effective at endotoxin removal, these affinity resins cannot be cleaned with standard depyrogenation methods (e.g., NaOH in ethanol). Anion exchange may also be used to remove a bulk of contaminating DNA from process streams, but may require a polishing step to reduce DNA to an acceptable level. Peptides with certain structural motifs have been shown to bind to DNA with high affinity and thus may be used for final host cell DNA removal. Contaminating metals from metal affinity chromatography (i.e., Ni-affinity chromatography) is another area of concern. Metals can act as oxidizers under certain conditions and can inactivate biopharmaceutical preparations by oxidizing certain residues such as histadine (His), tryptophan (Trp), and methionine (Met). Accordingly, removal of metal ions may be necessary in certain biopharmaceutical preparations to retain biological efficacy and may be accomplished via high affinity peptide ligands. For example, high affinity Ni-binding peptides may be used to facilitation metal ion removal. In addition to selectivity and affinity, another interesting feature of peptide ligands is that stability toward common biopharmaceutical sanitization methods can be designed into the structure.

TMA is an effective adsorbent for a variety of substances including metal. Phosphate specific peptide sequences attached to immunoresins have also been found to effectively reduce phosphate concentrations from solutions. The chemical structure for TMA is in Table 1 below, and includes a benzene ring with three carboxylic acid groups:

TABLE 1

| | |
|---|---|
| Chemical Name: | Trimesic Acid (TMA) |
| CAS No. | 554-95-0 |
| Chemical Structure | 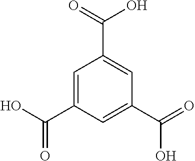 [SMILES: O=C(O)c1cc(C(=O)O)cc(C(=O)O)c1] |

TMA can readily complex with alumina to form a selective adsorbent complex specifically targeting adsorption of phosphate ions. In one embodiment, TMA that has been complexed with alumina can be chemically grafted to existing fabric 12 during adsorbent surface treatment 14 to create an adsorption device, or adsorbent fabric 10. TMA, both complexed with alumina and neat, can be grafted onto commercially available polyester filtration fabrics using any suitable process. In one embodiment, novel processes using heat and pressure, not limited to pressing in a Carver press, may be used to chemically graft TMA complexed with any suitable alumina, e.g., superbasic or activated alumina onto existing filtration fabric, fabric substrates, and/or available filtration cartridges. In another embodiment, novel thermal spray processes may be used to chemically graft TMA complexed with alumina or alumina alone onto existing filtration fabric and/or available filtration cartridges. In yet another embodiment novel atmospheric plasma-aided grafting processes can be used to chemically graft TMA with or without alumina onto existing filtration fabric and/or available filtration cartridges.

In one embodiment, adsorbent surface treatment 14 can comprise a novel heat and pressure process to graft TMA either alone or with alumina to filter fabric 12. FIG. 2 illustrates one embodiment of a pressing device, generally designated 20, for employing the heat and pressure process to transform existing fabric 12 to adsorbent fabric 10. In one aspect, pressing device 20 can comprise a Carver press and can have a housing 22 and at least first and second pressing plates 24 and 26, respectively. A filter fabric 28 may be pressed between pressing plates 24 and 26 in the direction indicated by the arrows indicating pressure P. Heat can be applied to a fabric substrate such as filter fabric 28 by pressing plates 24 and 26 as the Carver press can include a heating source or module for heating the pressing plates 24 and 26 to any suitable processing temperature. Materials that may be utilized during this pressurized and heated grafting process can include a suitable filter fabric 28, TMA, alumina powder, de-ionized water, methanol, polymer bags, various mixing and measuring containers, and pressing device 20.

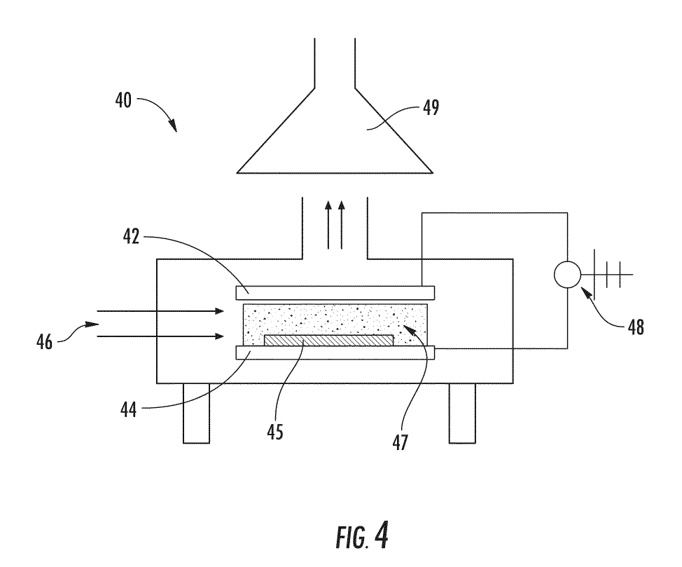

In one aspect, filter fabric 28 can comprise a PET medical grade filtration fabric. A first step in chemically grafting TMA with or without alumina to filter fabric 28 can be preparation of a TMA solution. The solution can be allowed to contemporaneously react while the filter fabric 28 is being loaded or coated with alumina (described further below). To prepare the TMA solution, 2.5 grams (g) of TMA can be added to 500 milliliters (mL) of methanol in a covered container. The 2.5 g of TMA may comprise 2.5 g of TMA pellets. The TMA and methanol can be sufficiently mixed using any process including but not limited to stirring, sonicating, shaking, or combinations thereof. A solution prepared using 2.5 g of TMA and 500 mL of methanol was used to treat filter fabric 28 swatches that were 6 centimeters (cm)×9 cm for data described in the Examples section and FIG. 5 further below. Any suitable ratio of TMA and methanol can be used. The solution should then be allowed to react, in one aspect, the solution can be allowed to react for approximately 6 hours before introducing the TMA solution to an alumina coated filter fabric 28. In one aspect, TMA can be reacted with alumina particles prior to the second step described below. That is, TMA may react with alumina particles, and the TMA complexed with alumina particles may then be pressed onto fabric as described further.

A second step to chemically graft TMA with alumina onto filter fabric 28 using pressing device 20 can comprise loading filter fabric 28 with alumina powder. This step can include a series of steps including cutting the fabric, coating the fabric with powder, and pressing the fabric in heated pressing device 20. Filter fabric 28 can be cut to any suitable or desired size and an initial mass can be measured and recorded. In the meantime, pressing device 20 can be pre-heated to 500 degrees Celsius (° C.). A suitable amount of alumina powder can be placed into a container. In this case, the TMA can be reacted with alumina after the alumina is pressed into the fabric. The amount of alumina powder to use depends on the size of filter fabric. For example, 5 g of alumina powder was used during experiments, as described in the Examples section below, for treating fabric swatches measuring 6 cm×9 cm. Filter fabric 28 can be immersed into de-ionized water and completely submerged for 10-15 seconds. Filter fabric 28 can then be removed from the de-ionized water and placed in the container of alumina powder to be sufficiently coated by the powder via shaking or stirring the fabric into the container of powder. Filter fabric 28 can be considered sufficiently coated when alumina powder substantially covers every surface of the fabric.

Still referring to FIG. 2, once filter fabric 28 has been coated by alumina powder, the alumina coated filter fabric 28 may then be placed between first and second pressing plates 24 and 26 of heated pressing device 20. Pressing plates 24 and 26 should be positioned in pressing device 20 such that the plates initially exert minimal pressure, for example, <1 kilopascal (kPa) on filter fabric 28. The pressure of pressing device 20 can then be increased such that filter fabric 28 becomes pressed between pressing plates 24 and 26 up to approximately 2000 pounds/square inch (PSI) for 20 seconds. Pressure may then be released and filter fabric can be removed and allowed to cool. The mass of the pressed filter fabric 28 can then be measured and recorded to calculate the amount of adsorbing complex that is loaded onto fabric. Fabric 28 can comprise any suitable mass of adsorbing complex. Filter fabric 28 can then be immersed in a clean container of fresh deionized water and the coating/pressing steps may then be repeated for any suitable number of times. That is, in one aspect the steps of immersing filter fabric 28, coating the filter fabric in alumina powder, and pressing the fabric in the heated pressing device 20 can be repeated at least two additional times, but is not limited thereto.

Upon pressing the alumina coated filter fabric 28 in pressing device 20, filter fabric 28 can be placed in a container and reacted with TMA solution. TMA solution can be poured over the fabric such that filter fabric 28 becomes completely immersed or submerged. Pressed, coated filter fabric 28 and reacted TMA solution may be covered and allowed to react for any suitable amount of time. In one aspect, coated filter fabric 28 and reacted TMA solution can be allowed to react in a covered container for approximately 6 hours. The fabric can then be removed and optionally subjected to a rigorous washing step which can include rinsing and/or scrubbing filter fabric 28 in de-ionized water for several seconds and can include multiple rounds of rinsing and scrubbing. In one aspect, rinsing alone can be used to remove unreacted chemical or ungrafted compound. Filter fabric 28 can then be dried and a final mass can be measured and recoded. The surface of filter fabric 28 which is chemically grafted with TMA and alumina and can be used alone and/or in combination with other devices to adsorb impurities, such as phosphates, from any suitable liquid including, but not limited, to blood and wastewater. Optionally, filter fabric 28 can be positioned and/or placed in a readily available filter cartridge used, for example, in dialyzers for dialyzing blood.

In a further aspect, adsorbent surface treatment 14 of FIG. 1 can comprise a thermal spray process for treating a surface or substrate of an existing fabric 12. FIGS. 3A to 3E are scanning electron microscope (SEM) images illustrating filaments of a fabric substrate such as filter fabric, generally designated 30, which have been grafted with alumina particles 32 using a thermal spray process. FIGS. 3A and 3B illustrate initial trials of thermally sprayed fabrics. FIGS. 3C to 3E illustrate thermal sprayed processes which were optimized, e.g., sprayed multiple times during experimentation to yield more particles 32 adhering to filter fabric 30. Thermally spraying of non-activated and/or activated alumina is contemplated. Grafting TMA and alumina particles using a thermal spray process can involve several steps. A first step can comprise introducing solid aluminum oxide powder feedstock into a high velocity, high temperature flame for approximately 1 millisecond (ms). During the approximate 1 ms that the powder particles are in the flame, they can become molten and accelerated towards the fabric substrate: As the accelerated particles impact the substrate, they can flatten and rapidly solidify in approximately 5-10 ms. This process may occur millions of times as the robot-mounted flame rasters across the substrate building up towards a final coating. FIGS. 3A to 3E illustrate various levels of coating ranging from lightly coated to heavily coated. In one aspect, the thermal spray process comprises sequential steps of spraying alumina particles onto existing fabric 12 and then treating, or reacting the sprayed fabric with a TMA solution. In another aspect, alumina particles can be pretreated with TMA prior to thermal spraying, such that TMA complexed with alumina particles becomes sprayed onto existing fabric 12.

The SEM images in FIGS. 3A to 3E illustrate interlaced fibers of a Polyethylene terephthalate filter fabric 30 and an aluminum oxide deposition 32. In one aspect, non-activated alumina comprises a low surface area and may not suitable for phosphate adsorption. To date, thermal spray techniques used to chemically graft alumina onto filter fabric comprise application of activated alumina onto the filtration fabric surface. In one aspect, activated alumina may be manufactured from aluminum hydroxide by dehydroxylating it in a way that produces a highly porous material. Activated alumina can comprise a surface area significantly over 200 square meters per gram (m/g), thereby rendering it advantageous for surface adsorption 30.

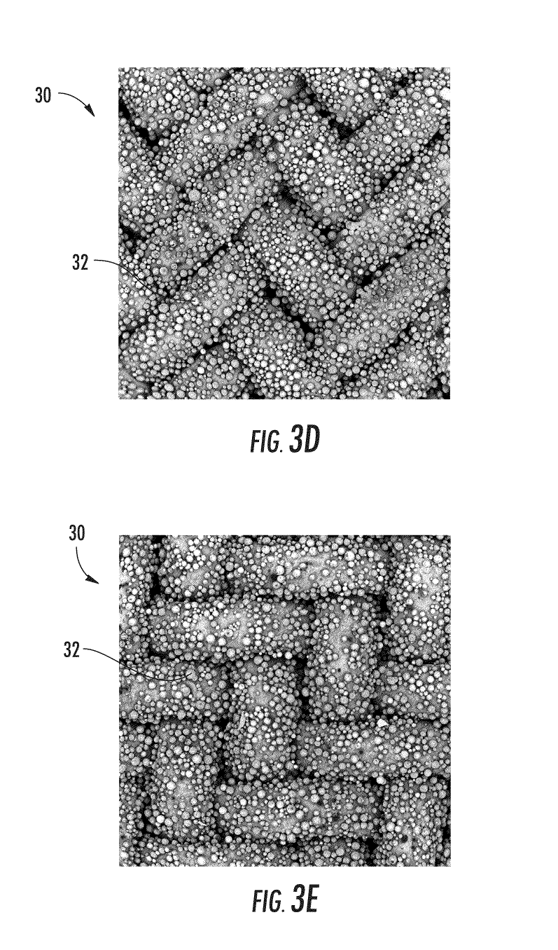

In a further embodiment, adsorbent surface treatment 14 can comprise a novel atmospheric plasma-aided grafting process for grafting TMA, or TMA complexed with alumina onto commercially available filter fabrics. FIG. 4 illustrates a plasma generating device, generally designated 40 for performing an atmospheric plasma surface treatment to prepare a surface of the fabric for accepting adsorbent chemical groups. A gas plasma pretreatment of an existing filter fabric may enhance the reactivity of fibrous substrates and aid in covalent bonding between the fabric and TMA and/or TMA complexed with alumina particles. In one aspect, grafting can be accomplished by interfacing the filter fabric with a prepared TMA or TMA and alumina solution subsequent to exposing the filter fabric to an atmospheric plasma surface treatment process. Plasma generating device 40 can comprise first and second electrodes 42 and 44. Plasma generating gas 46 can flow between the electrodes to establish a plasma bulk, generally designated 47 when subjected to electrical bias generated between electrodes 42 and 44 via power source 48. A fabric substrate 45 can be positioned between electrodes 42 and 44 such that the reactivity of the fibrous substrate can be enhanced when the substrate 45 is subjected to the atmospheric plasma surface treatment. Gas can then leave the plasma generating device through ventilation 49. A second plasma treatment subsequent to application of the TMA solution may also enhance the graft yield. One benefit of atmospheric plasma lies in the ability to achieve continuous manufacturing lines, and thereby, a high-throughput, economical, and environmentally benign production process can be established.

As noted above, a TMA and/or TMA with alumina solution can be applied to a filter fabric which has been pretreated in a plasma atmosphere. TMA and/or TMA with alumina solutions can be prepared by reacting a suitable amount of TMA pellets in methanol, and then optionally pouring the solution over alumina powder. In one aspect, fabric substrate 45 can comprise a medical grade filtration fabric, for example, a woven polyester monofilament, for example, manufactured by SaatiAmericas, PES of Somers, N.Y. Fabric substrate 45 can be pretreated for one or more minute(s) in a 99% helium, 1% oxygen capacitively-coupled dielectric barrier discharge atmosphere for creation of additional sites for covalent bonding of fabric finishes post-treatment. Other reactive gases may be added to He, or He may be used alone as the plasma medium. Immediately following the plasma pretreatment, the fabric can be dipped in the TMA and/or TMA/alumina solution. Fabric substrate 45 can then be optionally dried thereby evaporating the methanol. Fabric substrate 45 can then be retreated in a plasma system for 0.5, 1, 2, or 5 minutes (or any other duration) to graft the functional chemistry (TMA or TMA/alumina) to the surfaces of the fabric filaments. After grafting, fabric substrate 45 can be optionally rinsed in methanol for approximately 24 hours to remove excess non-reacted chemicals and/or particles. The fabric can be dried at room temperature or in an oven prior to use as an adsorbing device or used in an adsorbing system.

In one aspect, another phosphate adsorbing complex can comprise an aluminum hydroxide precipitate. Filtration fabric, for example, PET fabric can be treated in helium (He) plasma for approximately 5 minutes or more to improve wettability and surface functionality. Any suitable plasma composition can be used. Fabrics can then be placed into a vessel containing a solution of aluminum nitrate in deionized water. A base, e.g., sodium hydroxide or ammonium hydroxide, will be added to the solution to a pH of 9 in order to precipitate the aluminum hydroxide onto the fabric surface. For testing purposes, fabrics can be washed three times in de-ionized water, and heated in an oven at 70° C. for one hour.

### EXAMPLES

The following examples are provided, without limitation, for illustrating possible aspects and embodiments of the subject matter disclosed herein. However, those of ordinary skill in the art should, in light of the present disclosure, may appreciate that changes can be made in the specific examples and embodiments disclosed and still obtain a like or similar result without departing from the spirit and scope of the presently disclosed subject matter.

FIG. 5 illustrates a measured amount of phosphate (PO₄), e.g., 10 to 60 mg/L adsorbed over time in TMA-alumina grafted fabrics, the trial fabrics are labeled by trial number. The fabrics tested are commercially available, for example, woven monofilament filtration fabric manufactured by SaatiAmericas, PES of Somers, N.Y. Table 2 below includes at least a portion of the data corresponding to FIG. 5. TMA-alumina grafted fabrics have demonstrated the ability to adsorb approximately 10 to 68% of phosphate from a solution which has come into contact with the filter fabric.

TABLE 2
Results of phosphate adsorption assays for TMA-alumina grafted fabrics (with rigorous washing regimen employed)

| | | | | PERCENTAGE | | |
|---|---|---|---|---|---|---|
| | INITIAL | | | OF | | |
| | CALCULATED | DATE OF | MASS OF | PHOSPHATE | GRAFTING | |
| | PHOSPHATE | CARVER | PO₄ | ADSORBED | YIELD OF | ADSORPTION |
| FABRICS | CONCENTRATION | PRESS | ADSORBED | FROM INITIAL | FABRICS | CAPACITY |
| USED | (mg/L) | GRAFTING | (mg) | SOLUTION | (GRAMS) | (mg/L) |
| 4 | 10 | Aug. 27, 2010 | 0.629187945 | 68.71334119 | 0.0124 | 44.18537537 |
| 3 | 20 | Aug. 27, 2010 | 0.604237523 | 33.05458546 | 0.0222 | 23.74476651 |
| 1 & 2 | 30 | Sep. 2, 2010 | 0.814245722 | 33.86447557 | 0.0574 | 14.11279016 |
| 6 & 11 | 40 | Sep. 22, 2010 | 0.512078588 | 15.30365965 | 0.0277 | 17.62116785 |
| 3 & 5 | 50 | Sep. 15, 2010 | 0.868945705 | 22.29632396 | 0.0357 | 24.89970613 |
| 4 & 9 | 60 | Sep. 22, 2010 | 0.509199453 | 10.42395215 | 0.0292 | 17.07890313 |

Results of phosphate adsorption assays for various alumina powder formulations are in Table 3 below. Alumina powder has demonstrated the ability to adsorb approximately 99% of phosphate from an initial solution. Trimesic acid may be adsorbed onto alumina microparticles using previously described processes. Preliminary studies of TMA/alumina were conducted using standard basic alumina in activated form. Use of the “super” basic activated alumina (surface area 200 m²/g) can be used to boost adsorption.

TABLE 3
Results of phosphate adsorption assays for various alumina powder formulations

| | INITIAL | | | | |
|---|---|---|---|---|---|
| | CALCULATED | | | PERCENTAGE | |
| | PHOSPHATE | MASS OF | | OF | |
| | CONCEN- | TMA- | MASS OF | PHOSPHATE | |
| ALUMINA | TRATION OF | ALUMINA | PO₄ | ADSORBED | ADSORPTION |
| POWDER | SOLUTION | POWDER | ADSORBED | FROM INITIAL | CAPACITY |
| TYPE | (mg/L) | (GRAMS) | (mg) | SOLUTION | (mg/g) |
| BASIC | 30.00 | 1.00 | 2.564778621 | 99.44414833 | 2.38665956 |
| ACTIVATED | | | | | |
| SUPERBASIC | 30.00 | 1.00 | 2.435879125 | 99.95384717 | 2.398892332 |
| ACTIVATED | | | | | |
| NON- | | | | | |
| ACTIVATED | 30.00 | 1.00 | 0.107654289 | 4.163703279 | 0.099928879 |
| NEUTRAL | | | | | |

Adsorbent device 10 can also be transformed by reacting a selected set of peptide sequences with an existing fabric substrate, for example, polyester. In one aspect, selected sets of peptide sequences can be reacted either with polyester filter fabrics, or with a porous polymethylmethacrylate beaded resin bound to the fabric. The peptide sequences can be synthesized directly to the fabric or bound to the resin bead, or can be grafted to the fabric surface after synthesis. Commercially available resin beads with free amino groups can be chosen, and can provide excellent substrates for the synthesis of peptides by FMOC chemistry in an automated peptide synthesizer device (not shown). The peptide/resin linkage is covalent and after deprotection of the newly synthesized peptide by standard 95% trifluoroacetic acid treatment, the resin/peptide can be used directly as an immunoadsorbent. These resins, unlike the typical polystyrene resins used in solid phase peptide synthesis, are very water-friendly, allowing their use as immunoadsorbents in aqueous laboratory buffers.

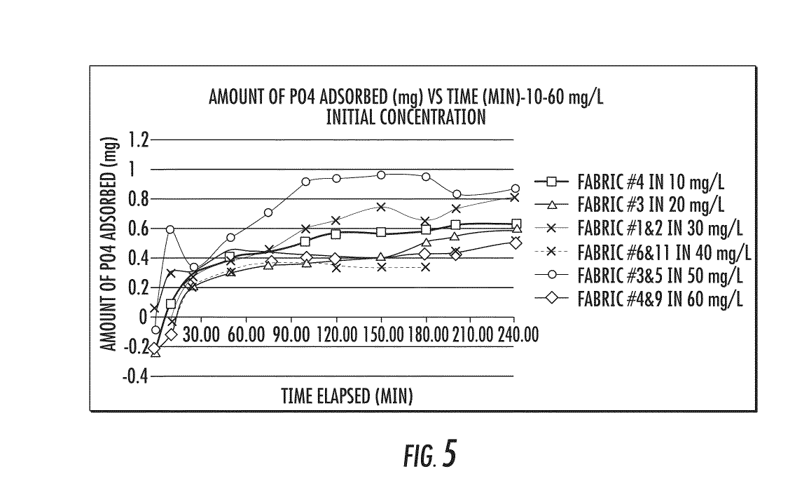

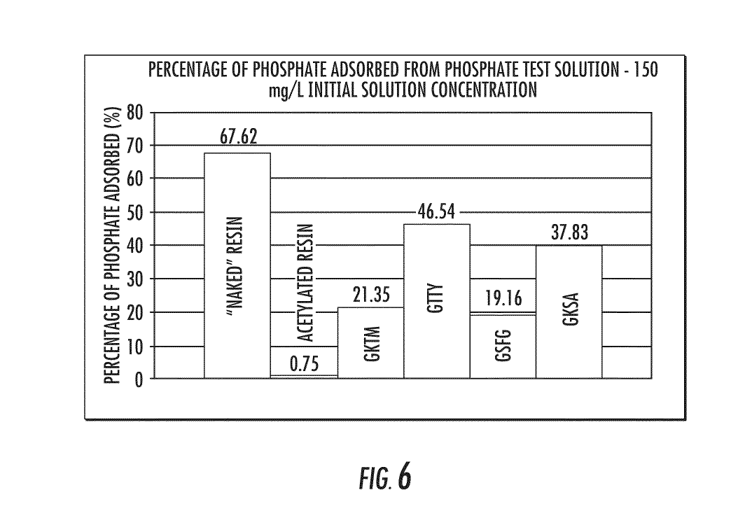

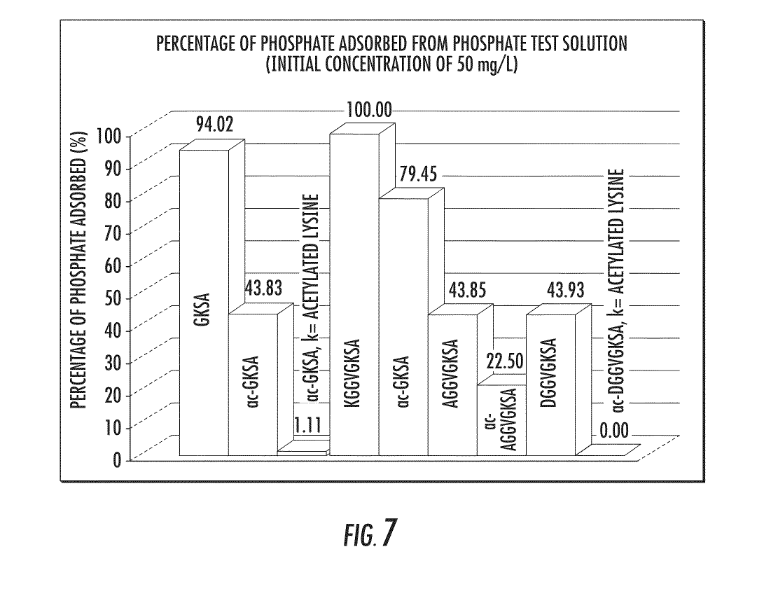

In one embodiment, selected tetra- and penta-peptides can be synthesized, purified, and certified using a multiple peptide synthesizer device. Sequences can optionally be synthesized prior to reaction with existing fabric 12. A second set of peptides containing not only the selected tetra- and penta-peptides, but also amino acid sequences located adjacent to these peptides in the native proteins, can be synthesized. Various results of the percentage of PO₄ adsorbed from phosphate test solutions using adsorption via peptide sequences is illustrated in FIGS. 6 to 8. For example, peptide sequences identified in Table 4 below summarizes results for peptide sequences with phosphate selective binding sites, the data of which is at least partially displayed in FIGS. 7 and 8.

TABLE 4
Collective results of phosphate assays on example synthesized peptide sequences and
respective adsorption capacities for each sequence at an initial phosphate concentration of 50 mg/L.

| | | INITIAL | | |
|---|---|---|---|---|
| SEQ. ID | PEPTIDE | CALCULATED | PERCENTAGE PO₄ | ADSORPTION |
| NO. | SEQUENCE | PO4 CONC. (mg/L) | ADSORBED | CAPACITY (mg/g) |
| 1 | KGGVGKSA | 50.00 | 100.00 | 4.31 |
| 3 | GKSA | 50.00 | 94.02 | 4.22 |
| 6 | AGGVGKSA | 50.00 | 82.82 | 3.56 |
| 2 | ac-KGGVGKSA | 50.00 | 79.44 | 3.42 |
| 8 | GKTM no surrounding | 50.00 | 87.58 | 3.40 |
| | Aa’s | | | |
| 3 | GKSA 1 | 50.00 | 86.65 | 3.20 |
| 3 | GKSA w/surrounding | 50.00 | 69.76 | 2.58 |
| | Amino Acids | | | |
| 9 | GTTY 1 | 50.00 | 54.48 | 2.01 |
| 4 | ac-GKSA | 50.00 | 43.80 | 1.96 |
| 10 | DGGVGKSA | 50.00 | 43.97 | 1.89 |
| 7 | ac-AGGVGKSA | 50.00 | 35.04 | 1.51 |
| 9 | GTTY w/surrounding | 50.00 | 26.31 | 0.97 |
| | Amino Acids | | | |
| 8 | GKTM w/Aa’s | 50.00 | 13.92 | 0.54 |
| 5 | ac-GkSA; k = | 50.00 | 1.11 | 0.05 |
| | acetylated lysine | | | |
| 11 | ac-DGGVGkSA; k = | 50.00 | 0.00 | 0.00 |
| | acetylated lysine | | | |

Table 4 above includes collective results of phosphate assays on selected synthesized peptide sequences and respective adsorption capacities for each sequence at an initial phosphate concentration of 50 mg/L. To clarify, common sequences share a sequence identification number, for example GKSA (Seq. ID No. 3) shares sequence identification with GKSA 1 (Seq. ID No. 3). The “1” identifies a separately synthesized batch number. The prefix “ac” refers to acetylated peptide sequences. The phosphate assays were performed after running phosphate solutions through a chromatography column loaded with a known amount of peptide-grafted resin beads.

In one embodiment, peptides may be synthesized on amino acids containing methacrylate based resin beads. As an example, FIG. 6 illustrates that approximately 46% of phosphate in this particular solution can be removed by specific peptide sequence (e.g., by GTTY, Seq. ID No. 9) as opposed to the non-specificity of phosphate removal with naked resin beads (67%).

FIGS. 6 through 8 illustrate the adsorption of peptide sequences synthesized onto resin beads. FIG. 6 illustrates results of testing synthesized peptide sequences synthesized on amino containing methacrylate based resin beads. Results indicate that variations of GTTY (Seq. ID No. 9) and GKSA (Seq. ID No. 3) have the greatest ability to adsorb phosphate. FIG. 7 illustrates results of phosphate adsorption assays on peptide sequences of GKSA (Seq. ID No. 3) variations. Results indicated that KGGVGKSA (Seq. ID No. 1) adsorbed 100% of phosphate from a test solution at an initial concentration of 50 mg/L. FIG. 8 illustrates collective results of phosphate adsorption on basis peptide sequences with and without surrounding amino acids. Any suitable peptide sequences are contemplated herein.

In one embodiment, peptide sequences used to generate the data illustrated by FIGS. 6 to 8 can be grafted neat or attached to resin beads and grafted onto commercially available polyester filtration fabrics, for example, a woven polyester monofilament medical grade filtration fabric. Amino acids require the presence of amine groups on the substrate surface for grafting. Gas plasma treatment and ethylene diamine (ED) aminolysis can create amine sites for grafting the protein onto the polyester surfaces. Gas plasma pretreatment may enhance the reactivity of fibrous substrates and aid in covalent bonding of surface treatments. In one aspect, fabric samples can be treated for approximately five minutes in approximately 99% helium and 1% forming gas (a mixture of hydrogen (up to 5.7%) and nitrogen) capacitively-coupled dielectric barrier discharge. Any suitable plasma composition may be used. This treatment can create additional sites for covalent bonding of fabric finishes post-treatment. Fabric samples can be placed in a 0.05 mole (M) ED solution at pH 10 for approximately two hours instead of the gas plasma treatment. The samples can be rinsed in ice-cold de-ionized water to remove excess ED solution and halt the reaction. The samples can be dried at room temperature in a fume hood. After surface modification, the fabric can be grafted by dipping into a solution of the peptide adsorbent chemistry, and nipping to remove excess chemical. The fabric samples can be thoroughly rinsed in a solvent for 24 hours to remove excess non-reacted chemicals/particles and allowed to dry at room temperature in a fume hood. Fabrics can be weighed before and after treatment to determine the weight gain due to chemical grafting.

In one embodiment, the starting resin for peptide synthesis may include an amino containing methacrylate based resin. Peptides can first be synthesized on amino containing methacrylate based resin beads. Peptides can be synthesized with a carboxyl terminal di-alanine on the resin in order to keep the peptide of interest slightly away from the resin surface and free to adopt a unique conformation with some freedom of movement. Amino-loaded resins, for example as manufactured by Tosoh, can be very water-friendly and the surface amino group allows for peptide synthesis directly on the resin with an acid stable, covalent linkage, permitting these resins to be reused multiple times. These resins allow for peptide synthesis at the millimole scale and allow for the rapid evaluation of phosphate binding ability under a variety of conditions. As one form of control, a positively charged amino acid (lysine and/or arginine) instead of the tetra- or penta-peptides can be prepared on the surface of the resin. The peptide-bead products can be prepared for affinity columns by washing with water and mixing with water to make 30-40% slurry.

The amount of phosphate bound to chromatography column versus in solution flow-through allows selection of the most efficient peptides for subsequent phosphate binding experiments. The ability to synthesize peptides directly on the polyester filament has not been previously demonstrated. An alternative strategy is to synthesize the sequence prior to binding it to the fabric. Additionally, the resin beads have much higher surface area than the polyester fabric alone; therefore, in order to maximize phosphate adsorption the method used for fixation of the TMA/AI complex onto the polyester filtration fabrics can be replicated for the treated beads.

For testing phosphate adsorption, for example, testing to generate FIG. 5, TMA grafted textile fabric samples can be cut into approximately 0.5 g swatches. Swatches were placed into 100 mL screw capped vials containing 50 mL of phosphate solutions, for example, a Ringer's Lactate solution of different phosphate concentrations. Fabric samples were withdrawn from the phosphate solution at testing times ranging from 10 to 240 minutes and the amount of phosphate adsorbed was calculated. For peptides, the phosphate assays were performed after running phosphate solutions through a chromatography column loaded with a known amount of peptide-grafted resin beads. In one embodiment, the adsorption capacity for phosphate (q) will be calculated using Equation 1:

q(mg/g)=[Amt PO_{4 initial}−Amt PO_{4 time-x}]/[mass gain of fabric due to grafting] EQUATION 1:

This calculation allows graphs of adsorption efficiency (%) versus time to be constructed. Adsorption isotherms can also be plotted, and data analyzed with a Langmuir isotherm using Equation 2:

qₑ=(QₘbCₑ)/(1+bCₑ) EQUATION 2:

In this equation, qₑ is the adsorptive capacity at equilibrium, Cₑ is the equilibrium phosphate concentration, Qₘ is the maximum uptake capacity, and b is the Langmuir binding constant. Different regression methods can be used to evaluate the fit of the Langmuir model to adsorption data as this will enable differentiation of the least biased parameter estimates.

### Non-Limiting Applications of Adsorbent Devices and Systems

FIGS. 9-12 illustrate various non-limiting applications for adsorbent devices, systems, and methods described herein. FIG. 9 illustrates a system, generally designated 50 for treating a contaminated source 52, for example, wastewater, water, bodily fluids or blood. Any suitable contaminated source 52 is contemplated. Contaminated source 52 can be transformed into a filtered product 54 when the contaminated source 52 passes through or across adsorbing fabric 56. Adsorbing fabric 56 can contain adsorbent complexes 58 attached thereto for selectively binding to undesirable substances, for example, phosphate ions in contaminated source 52. In one aspect, adsorbent complexes 58 can be TMA, with or without alumina, alumina alone, synthesized peptide sequences, or aluminum hydroxide precipitates as previously described. Adsorbent complexes 58 may selectively and permanently adsorb undesirable substances, for example, undesirable concentrations of phosphate ions from contaminated source 52. For illustration purposes, adsorbent complexes 58 are schematically illustrated as visible complexes. However, these complexes 58 may or may not be particles. In one embodiment, adsorbent complexes 58 can comprise chemically reactive substances invisible to the naked eye or even microscopy. Adsorbent complexes 58 can comprise molecular compounds grafted or absorbed onto the surface of the fabric or other substrate.

FIG. 10 illustrates a hemodialysis system or circuit with addition of a hemoadsorbent device, the system generally designated 60 for removing phosphates from blood. Currently, there are no available phosphate selective blood filtration fabrics on the market which can selectively remove phosphates. Devices disclosed herein can comprise a whole blood filtration fabric that selectively adsorbs blood phosphates onto its surface through high capacity binding and can be integrated into a commercially available filter device or system. Existing devices that provide some filtration, e.g., hemodialyzers, can remove some phosphates through simple diffusion. The level of phosphate binding to the treated fabric can be tailored in the adsorbent device based on the ratio of adsorbent chemical groups to surface area. The design takes advantage of the existing whole blood filtration knowledge base and employs a commercially available filtration fabric that is designed for minimal blood cell adhesion or damage. The advantages over existing filtration devices and their processes include, for example and without limitation, the mechanism of phosphate removal (adsorption vs. simple diffusion), the prospect of prescribing a phosphate filtration dose, and the ability to remove larger phosphate loads. As an overall long-term outcome, improved phosphate control and its associated reduction in systemic phosphate burden may lead to reductions in hemodialysis patient mortality.

Referring to FIG. 10, hemodialysis circuit or system 60 can comprise an extracorporeal blood circuit for dialysis of blood from a hemodialysis patient. An in-line hemofiltration/hemoadsorption device for hyperphosphatemic hemodialysis subjects or patients is contemplated. System 60 can comprise various devices positioned along a circuit for processing blood, the devices being connected by one or more strands of blood tubing 62 for continuously flowing blood through system 60. Blood may be continuously extracted from a patient's body, for example, extracted from the subject's arm A into blood tubing 62, and continuously flow about system 60 in a direction indicated by the arrows to be processed and/or monitored using one or more devices. Processed blood can then be continuously fed back into the patient's body using blood tubing 62.

System 60 can comprise one or more pressure monitors 64 distributed at various points throughout the circuit for monitoring various pressures associated with blood flowing through the circuit. For example, a first pressure monitor 64 of system 60 can be positioned along blood tubing 62 proximate to where the blood is extracted from arm A to measure arterial pressure. Arterial pressure can be monitored either prepump (as shown) or postpump depending on the type of hemodialysis machine and blood tubing being used. The pressure readings for prepump versus postpump arterial monitors may provide different information regarding the hemodialysis treatment and/or the patient's access. For example, arterial vascular access and clotting problems may be identified using first pressure monitor 64 of system 60.

System 60 can comprise a blood pump 66 for controlling the circulation of blood within system 60. Blood flowing along the hemodialysis circuit can be treated using a heparin pump 68 employed to inject heparin into blood leaving the body before passing through a dialyzer 70 for preventing the blood from clotting. Dialyzer 70 subjects the blood to dialysis, and can include any suitable type of dialyzer. For example, dialyzer 70 can comprise a coil type, flat plate-type, laminate type, hollow fiber, a continuous hemofilter, or any type of dialyzer as known in the art which may or may not utilize a dialysate. In one embodiment dialyzer 70 is configured for continuously flowing blood and a dialysate through the dialyzer at the same time. In one embodiment, dialysate may flow through the dialyzer 70 from a lower passage 73 to an upper passage 71. Dialysate can absorb waste products, and then drain out through the upper passage 71 to be discarded. As known in the art, dialysate and blood may flow in opposite directions in a blood dialyzer system.

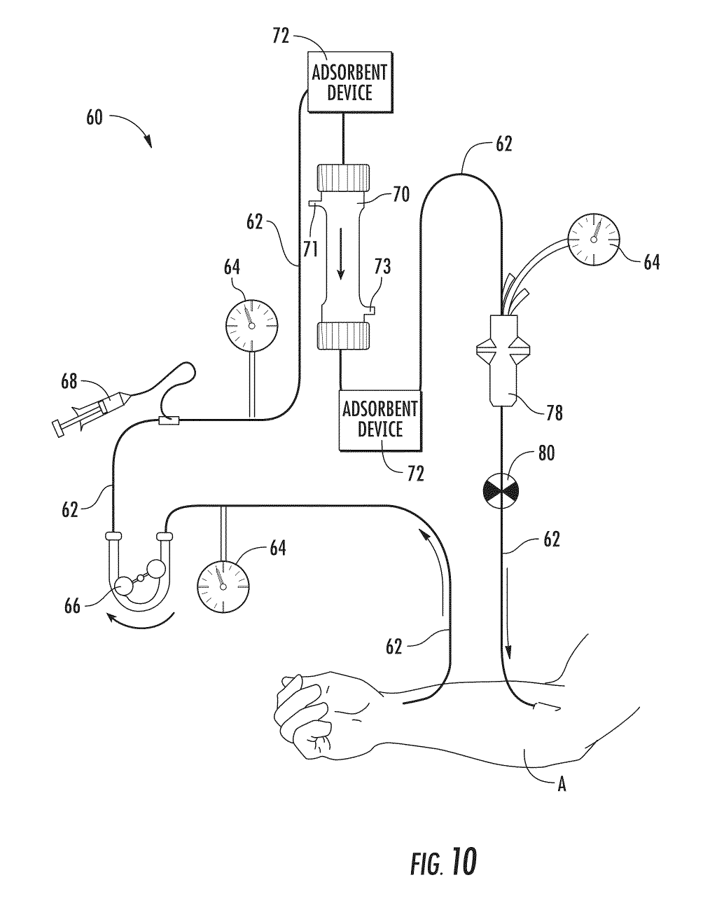

Notably, adsorbent devices 72 in accordance with the novel subject matter described herein can be positioned upstream and/or downstream of dialyzer 70 for processing the blood either before or after the blood passes through dialyzer 70 to undergo dialysis. In one embodiment, adsorbent devices 72 can comprise standalone devices (FIGS. 11 and/or 12) or may be positioned within dialyzer 70 (FIG. 10) either as a separate portion or integrally formed with dialyzer 70 such that blood can pass through adsorbent devices 72 simultaneously with dialyzer 70. Adsorbent device 72 and dialyzer 70 can be configured for removing substances or impurities from blood and/or dialysate contacting adsorbent device 72. In one aspect, adsorbent device 72 can comprise a filter fabric which has been chemically grafted with a phosphate adsorbing complex for selectively removing phosphate from the blood. Once the blood passes through adsorbent device 72 and dialyzer 70 for purification or from dialyzer 70 to adsorbent device 72, it can then pass through an air trap 78 and detector 80 before being continuously fed back into arm A of a patient.

Fabric samples treated with adsorbing complexes described herein may be tested in a blood circuitry system (e.g., FIG. 10). FIG. 10 illustrates a typical human hemodialysis circuit that may be evaluated when the addition of an adsorbent fabric device is used. In hemodialysis circuits, arterial blood can be driven by a pump through the dialyzer and then return to the patient as venous blood. The circuit can be tested using a reservoir of porcine whole blood prepared to mimic ESRD blood phosphate concentrations of 10 to 50 mg/L and passed through adsorbent devices. The blood can be maintained at physiological pH (7.4) and temperature (37° C.) during the experiments and circulated by a variable flow peristaltic pump, for example, a pump manufactured by Fisher Scientific, Pittsburgh, Pa. A constant heparin infusion of 150 U/hr can be maintained via an infusion pump, for example, a pump manufactured by KD Scientific, Holliston, Mass. to prevent clotting in the dialyzer and phosphate hemofilter. A standard bicarbonate dialysate solution containing potassium and calcium can be prepared and pumped through the dialyzer by a variable flow peristaltic pumped in a direction countercurrent to the direction of blood flow. Blood samples can be collected before and after passage through the hemoadsorption device (for example, 72 of FIG. 10) at times 0, 30, 60, 120, 180, and 240 minutes, or any suitable time intervals. Adsorption data from the studies can be calculated.

One embodiment of an adsorbent device, generally designated 90 is illustrated in FIG. 11. Adsorbent device comprises an inlet 92 and an outlet 94 through which any suitable contaminated liquid can pass. Adsorbent device 90 can comprise a filter fabric 96 that has been treated with an adsorbing complex. Filter fabric 96 can be enclosed in a device housing 98. The enlarged view of filter fabric 96 illustrates fabric filaments 100 with adsorbing complexes 102 attached thereto. Adsorbing complexes 102 can be chemically grafted or synthesized to filaments 100 of fabric, or any other suitable substrate. Adsorbent device 90 can comprise any suitable size or shape and orientation having inlet 92 and outlet 94 through which a liquid may pass. Adsorbent device 90 can be configured for filtering blood, water, wastewater, bodily fluids or any liquid in which removing impurities or contaminants is desired.

In one aspect, adsorbent device 90 can be used to selectively filter blood to prevent and treat hyperphosphatemia in patients suffering from ESRD. For example, adsorbent device 90 could be positioned upstream and/or downstream of a dialyzer, for example dialyzer 70 (FIG. 10) for selectively removing materials such as phosphate from the blood before or after the blood is dialyzed. The adsorptive agent or complex bound to the filter fabric surface can be designed to selectively remove phosphate ions with little to no effect on the concentrations of other blood electrolytes or other blood chemistry compounds. Other phosphate filtration technologies may include ion exchange resins and non-selective adsorbents such as carbon. Ion exchange resins act via chemical reactions that consume phosphate and release other blood electrolytes, thereby, altering blood chemistry in a potentially dangerous manner, particularly relevant to hemodialysis patients who often have underlying electrolyte and acid-base abnormalities. Non-selective adsorbents may rely on physical, rather than chemical adsorption of ions in microscopic pores. Activated carbon is an excellent adsorbent with very high surface to volume ratios, but is not ion specific. If trace metal concentrations are adversely affected by adsorbent device 90 or an alternative adsorbent, they could be added back into the bloodstream during or after dialysis. Adsorbent device 90 can comprise a stand-alone single or multiple use in-line hemofiltration device that provides selective blood phosphate adsorption. It can comprise an off-the-shelf device that is easily integrated within existing hemodialysis circuitry (FIG. 10) and requires no additional treatment or monitoring equipment. Adsorbent device 90 comprises unique phosphate selective absorptivity to provide significant reduction in blood phosphate levels beyond what is offered by dialysis alone or in combination with controlled diet or drugs. This novel device can provide rapid deployment, ease of use, and simple disposal. Blood can be pumped through the filter 96 at the typical rates prescribed during hemodialysis (500 to 800 mL/min) and will pass directly back into the patient, with no additional filtration or treatment.

In one aspect, adsorbent device 90 can comprise a surface enhanced filtration fabric 96 formed into a cartridge, and housed within a clear plastic filter housing 98. Devices and systems described herein can comprise, for example, three main components that can be: an adsorbent fabric 12, a medical grade filter fabric with a phosphorus-selective adsorbent finish 100, the filter cartridge form and the plastic filter housing unit 98; and a filter integrated within a filter system, such as a dialyzer 70 which is a single unit. Filters described herein can be easily substituted into existing filter housings and can provide rapid implementation of phosphate binding technology at a low cost. Additionally, the filters described herein have capability of being removed, cleaned, and sterilized for subsequent reuse. Regarding device 90, there are no parts to assemble. The entire unit is self-contained and designed to interface with existing dialysis circuitry and systems. Blood can be pumped at the typical rates prescribed during hemodialysis (500 to 800 mL/min) through the filter, and will pass directly back into the patient, with no additional filtration or treatment.

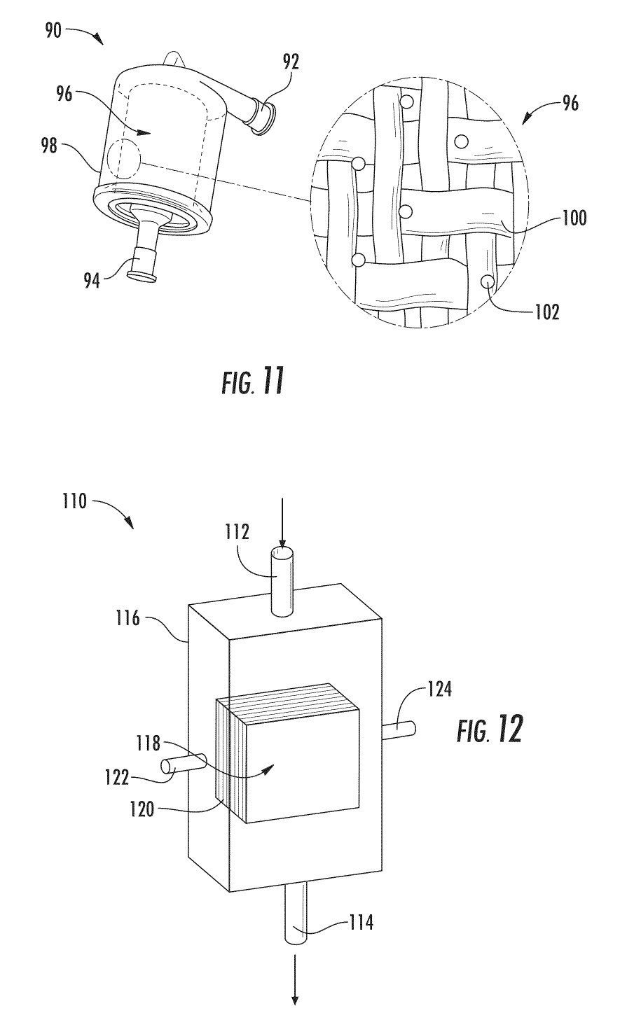

FIG. 12 illustrates a filter system, generally designated 110. In one aspect, filter system 110 filters blood. System 110 can include an inlet 112 and outlet 114 through which a liquid can flow in the direction indicated by the arrows. System 110 can include a housing 116 surrounding a filter cartridge, or filter 118. Surfaces of filter 118 can have been previously treated with an adsorbing complex such that undesirable ions or particles can be adsorbed thereto. In one embodiment, filter system 110 comprises an adsorbent device of a flat plate design. It may comprise a stand-alone device similar to that described with respect to device 90. System can comprise a filter cartridge 118 and housing 116.

In a second embodiment, system 110 can comprise a dialyzer having an integral filter 118 attached thereto, thus, it could be used as dialyzer 70 in the circuit described in FIG. 10. Filter system 110 may comprise any suitable dialyzer known in the art including but not limited to a flat plate dialyzer, a continuous hemofilter, or a hollow fiber dialyzer. Dialysis works on the principles of the diffusion of solutes and ultrafiltration of fluid across a semi-permeable membrane. Blood can flow by one side of a semi-permeable membrane, and a dialysate, or special dialysis fluid, can flow by the opposite side. In one aspect, system 110 comprises inlet and outlet passages 122 and 124 for flowing a dialysate which removes waste products present in the blood by diffusion gradient. However, such passages 122 and 124 are optional and may not be required. Filter 118 may comprise a semi-permeable fabric membrane which has been chemically grafted or reacted to receive adsorbing complexes, such as phosphate adsorbing complexes. Filter 118 can selectively adsorb phosphate from blood and/or dialysate which contacts the fabric to remove the phosphate.

In one aspect, filters 96 and 118 can comprise a polyester filtration fabric formed into a cartridge treated using any method described earlier to chemically graft adsorbing complexes such as TMA, alumina, or adsorbing proteins and peptide sequences onto the surface of the polyester. Adsorbent (TMA alone, TMA/alumina, alumina alone) can be grafted as determined and inserted into its plastic housing. The filter units can be incorporated into the hemodialysis circuitry for selectively adsorbing phosphate during hemodialysis. Filter 118 can be incorporated into an apparatus at least similar in physical design to flat plate dialyzers known in the art, to enable enough of the treated fabric to be used in a device or system.

Selective adsorption of phosphates has been explored as a means to address environmental concerns regarding wastewater treatment. Certain organic compounds have been shown to be effective adsorbents for a variety of metal ions. A number of metal oxides form strong complexes with these compounds and can be used to provide a solid support for the chemistry. These complexes have been used to achieve selective phosphate filtration for effluent. Phosphate adsorbing complexes may be permanently fixed on the surface of medical grade polymers and can achieve optimum adsorptive capacity. Control of phosphate adsorption is tunable according to the ratio of adsorbent chemical groups to filter fabric surface area. Thus, adsorbent filters 96 or 118 can comprise different adsorptive “strengths”. The substrate fabric can be designed for minimal blood cell adhesion or damage. Since dialyzed blood is treated with anticoagulating pharmaceuticals (e.g. heparin), minimal effects of blood contact activation or thrombogenesis are expected upon perfusion through the fabric. Preliminary data upon exposure of heparinized blood to the fabric for four hours failed to demonstrate any evidence of thrombogenesis.

In one embodiment, adsorbent complexes or agents bound to the surface of filters 96 and 118 can be designed to selectively remove only phosphate ions and not alter the concentrations of blood electrolytes. Other phosphate filtration technologies include ion exchange resins and non-selective adsorbents such as carbon. Ion exchange resins act via chemical reactions that consume phosphate and release other blood electrolytes, thereby, altering blood chemistry in a potentially dangerous manner, especially since hemodialysis patients often have underlying electrolyte and acid-base abnormalities. Non-selective adsorbents rely on a physical, rather than chemical adsorption of ions in microscopic pores. For example, activated carbon is an excellent adsorbent with very high surface to volume ratios, but is not ion specific. Filters 96 and 118 can comprise a robust absorptive surface that does not rely on ion exchange, but rather, is a simple molecular association of phosphate with the adsorbent that occurs at normal blood pH (7.4) and temperature. An example of a bound adsorbent is TMA, alone or complexed with alumina particles, or alumina particles alone. Other bound adsorbents include any suitable peptide sequence or aluminum hydroxide or aluminum hydroxide precipitate. The adsorbent may be grafted via atmospheric plasma treatment either before, after, or both before and after exposure of the fabric substrate to a solution containing the adsorbent chemistry. Any suitable process for binding adsorbents is envisioned in accordance with the disclosure herein.

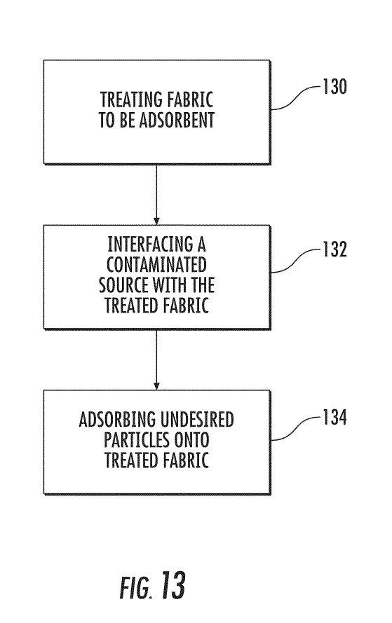

A method of filtering a contaminated liquid is illustrated in FIG. 13. The method includes treating a fabric substrate to be adsorbent. This can include chemically grafting TMA with or without alumina particles or synthesizing peptide sequences onto polyester or any suitable fabric. In one aspect, thermal spraying can be used to treat a fabric. In another aspect, a pressing device can be used. In yet other aspects, peptide synthesizers and atmospheric plasma can be used to treat fabric such that it adsorbs unwanted substances from an aqueous solution. The method can further comprise interfacing a contaminated source with the treated fabric at block 132. The contaminated source can be pumped, passed, or submerged in the treated fabric. Any suitable method of interfacing a liquid with the treated fabric can be used. The method can further comprise adsorbing undesired particles or ions onto the treated fabric at block 134. Undesired particles can be adsorbed onto the adsorbing complex on the surface of the treated fabric. In one aspect, TMA, with or without alumina, can be used to adsorb phosphate from blood or wastewater. Similarly, peptide sequences comprising phosphate binding proteins can be used to adsorb phosphate from blood or wastewater.

In sum, application of selectively adsorbent surface chemistry to textile-based filter materials can be adapted for adsorption of non-selective or other selective agents. Methods described herein can utilize a series of wet chemical and/or atmospheric plasma treatments to prepare fabric surfaces for acceptance and binding of the adsorbent complexes, as well as heat treatments to permanently bond the complexes. The adsorbing complexes can consist of either TMA bound to Alumina particles, or selected amino acid sequences either neat or bound to polymethylmethacrylate resin beads. Gas plasma pretreatment can enhance the reactivity of fibrous substrates and aid in covalent bonding or grafting of compounds to polymer substrates by creating either chemically active substituents or free radicals. In one aspect, textile-based filter materials that have been grafted with adsorbing particles can be used to filter phosphates from blood. In other aspects, filtration materials can be used for treatment of sepsis/septic shock and acute liver failure. Phosphates in domestic wastewater represent a major source of organic pollution in bays and lakes. Biological treatment processes are ineffective in solid phosphate removal, requiring additional selective phosphate filtration techniques. Additionally, phosphate recovery from wastewater is a potential solution to the rapid depletion of world phosphate resources. Treated, or grafted fabrics disclosed herein can offer a simple and innovative solution to this pressing environmental challenge.

Adsorbent devices, systems, and methods described herein provide benefits to physicians, patients, and the environment. Physicians are held accountable for having their patients successfully reach blood phosphate targets. The devices and systems described herein can improve patient outcomes from both cardiovascular and nutritional standpoints and will enable physicians to concentrate on other health concerns. Incorporation of adsorbing fabrics into dialysis devices and/or systems can result in significant cost reductions for patients given the dramatic improvement in compliance and significant reduction and better management of blood phosphate levels. The cost benefit includes reductions in costs for phosphate binding drugs and vitamin D supplements, as well as reductions in total. Hospitalizations due to phosphate levels can also be avoided. A potential “green” feature of devices and systems described herein is reuse of TMA/alumina treated fabrics, since the phosphate binding may be reversed by adjusting pH to higher values, i.e., by washing with a basic solution (e.g. sodium hydroxide; pH 10). Another key benefit of the technology described herein is improved patient compliance. This results from the incorporation of adsorption devices and systems described herein into the thrice weekly hemodialysis sessions in tandem with the dialysis procedure. Serum phosphate levels will not be dependent upon difficult-to-control factors such as patient compliance with dietary restrictions and medications.

Embodiments of the present disclosure shown in the drawings and described above are examples of numerous embodiments that can be made within the scope of the appended claims. It is contemplated that the configurations of adsorption devices, systems, and methods can comprise numerous configurations other than those specifically disclosed herein.

What is claimed is:

1. An adsorption device for reducing a level of phosphate in blood, the device comprising:
a filter fabric; and
an adsorbing complex attached at least partially to a surface of the fabric, the adsorbing complex being reactive to selectively adsorb phosphate from blood filtered through the fabric to remove phosphate from the blood.

2. The device of claim 1, wherein the adsorption device is adapted for filtering phosphate from the blood without altering chemistry of the blood.

3. The device of claim 1, wherein the adsorbing complex comprises trimesic acid (TMA).

4. The device of claim 3, wherein the adsorbing complex further comprises alumina.

5. The device of claim 1, wherein the adsorbing complex comprises a phosphate binding protein.

6. The device of claim 5, wherein the phosphate binding protein comprises a peptide sequence comprising a phosphate binding domain.

7. The device of claim 6, wherein the phosphate binding protein is bound to a resin bead.

8. The device of claim 7, wherein the adsorption device comprises a hemoadsorption device adapted to filter phosphate from blood in an extracorporeal blood circuit.

9. The device of claim 1, wherein the fabric comprises a filtration fabric selected from the group consisting of polyester fabric, non-polyester fabric, woven polyester monofilament, polymer and filament, or a combination thereof.

10. The device of claim 1, wherein the adsorbing complex comprises alumina or aluminum hydroxide.

11. The device of claim 1, wherein the adsorbing complex is reactive to selectively adsorb phosphate from dialysate flowing through a dialyzer.

12. A method of providing an adsorption device, the method comprising:
providing a fabric; and
grafting an adsorbing complex to a surface of the fabric, the adsorbing complex being reactive to selectively adsorb a substance from an aqueous solution.

13. The method of claim 12, wherein grafting an adsorbing complex to the surface of the fabric substrate comprises:
loading the fabric with alumina powder;
pressing the loaded fabric in a heated pressing device; and
immersing the pressed fabric in a trimesic acid (TMA) solution.

14. The method of claim 12, wherein grafting the adsorbing complex to the surface of the fabric substrate comprises grafting an adsorbing protein onto the fabric using a gas plasma treatment or ethylene diamine (ED) aminolysis adapted to create amine sites for grafting the protein onto the fabric surface.

15. The method of claim 12, wherein grafting the adsorbing complex to the surface of the fabric substrate comprises synthesizing a phosphate binding protein directly onto the surface of the fabric substrate.

16. The method of claim 12, wherein grafting the adsorbing complex to the surface of the fabric substrate comprises binding a phosphate binding protein to a resin bead.

17. The method of claim 12, wherein grafting the adsorbing complex to the surface of the fabric substrate comprises thermally spraying alumina particles onto the fabric substrate.

18. The method of claim 12, wherein grafting the adsorbing complex to the surface of the fabric substrate comprises treating the surface of the fabric using atmospheric plasma and exposing the fabric to a trimesic acid (TMA) solution.

19. A filtering device provided by the method of claim 12.

20. A method of treating blood, comprising:
providing a hemofiltration device comprising a filter at least partially constructed of a fabric, wherein an adsorbing complex has been grafted to a surface of the fabric; and
interfacing the blood with the hemofiltration device whereby a substance is selectively removed from the blood when the blood passes through the filter.

21. The method of claim 20, wherein the substance comprises phosphate that is selectively removed without substantially affecting other blood chemistry compounds.

22. The method of claim 20, wherein the filter comprises a filter cartridge.

23. The method of claim 22, wherein the filter cartridge is reusable.

24. The method of claim 20, wherein the fabric is selected from the group consisting of polyester fabric, non-polyester fabric, woven polyester monofilament, polymer and filament, or a combination thereof.

25. The method of claim 24, wherein the adsorbing complex is grafted to the surface of the fabric via a thermal spray process.

26. The method of claim 24, wherein the adsorbing complex is grafted to the surface of the fabric via a pressing process.

27. The method of claim 24, wherein the adsorbing complex comprises a phosphate adsorbing protein that is grafted to the surface of the fabric via a peptide sequence synthesizing process.

28. The method of claim 27, wherein the peptide sequence synthesizing process comprises using a plasma treatment or ethylene diamine (ED) aminolysis process adapted to create amine sites for grafting the phosphate adsorbing protein onto the fabric surface.

29. A method of treating blood, the method comprising:
providing an adsorbing device comprising:
a filter fabric; and
an adsorbing complex attached at least partially attached to a surface of the fabric;
positioning the adsorbing device in an extracorporeal blood circuit; and
interfacing blood with the adsorbent device for selective removal of phosphate from the blood.

30. The method of claim 29, wherein the adsorbing device is positioned upstream from a dialyzer.

31. The method of claim 29, wherein the adsorbing device is positioned downstream from a dialyzer.

32. The method of claim 29, wherein the adsorbing device is formed integral with a dialyzer.

33. The method of claim 32, wherein the dialyzer comprises a flat-plate type dialyzer.

34. The method of claim 31, wherein the dialyzer comprises a hollow fiber type dialyzer.

35. The method of claim 32, wherein the dialyzer comprises a hemofilter.

36. An adsorbent system comprising:
an adsorbent device comprising:
a fabric; and
an adsorbing complex attached at least partially to a surface of the fabric, the adsorbing complex being reactive to selectively adsorb a substance from an aqueous solution; and
a housing disposed about the adsorbent device.

37. The system of claim 36, wherein the system is adapted to selectively adsorb phosphate from the aqueous solution.

38. The system of claim 36, wherein the system is adapted for filtering phosphate from blood without altering chemistry of the blood.

39. The system of claim 36, wherein the adsorbing complex comprises trimesic acid (TMA).

40. The system of claim 39, wherein the adsorbing complex further comprises alumina.

41. The system of claim 36, wherein the adsorbing complex comprises a phosphate binding protein.

42. The system of claim 41, wherein the phosphate binding protein comprises a peptide sequence comprising a phosphate binding domain.

43. The system of claim 41, wherein the phosphate binding protein is bound to a resin bead.

44. The system of claim 36, wherein the adsorbing complex comprises alumina.

45. The system of claim 36, wherein the housing comprises a dialyzer.

46. The system of claim 36, wherein the fabric comprises a filtration fabric selected from the group consisting of polyester fabric, non-polyester fabric, woven polyester monofilament, polymer and filament, or a combination thereof.

47. The system of claim 36, wherein the adsorbing complex comprises aluminum hydroxide.

48. The system of claim 36, wherein the adsorption system is adapted to filter phosphate from dialysate in an extracorporeal blood circuit.

49. An adsorption device comprising:
a fabric; and
an adsorbing complex attached at least partially to a surface of the fabric, the adsorbing complex being reactive to selectively adsorb a substance from a liquid passing through the fabric to remove at least a portion of the substance from the liquid.

50. The device of claim 49, wherein the substance comprises phosphate.

51. The device of claim 49, wherein the adsorbing complex comprises trimesic acid (TMA).

52. The device of claim 51, wherein the adsorbing complex further comprises alumina.

53. The device of claim 49, wherein a peptide sequence is grafted to the fabric for selective organic ion adsorption.

54. The device of claim 49, wherein the adsorbing complex comprises a phosphate binding protein.

55. The device of claim 54, wherein the phosphate binding protein comprises a peptide sequence comprising a phosphate binding domain.

56. The device of claim 55, wherein the phosphate binding protein is bound to a resin bead.

57. The device of claim 49, wherein the adsorption device is adapted for filtering phosphate from blood without altering chemistry of the blood.

58. The device of claim 49, wherein the adsorption device is incorporated into an extracorporeal blood circuit adapted to filter phosphate from blood of a hemodialysis patient.

59. The device of claim 49, wherein the fabric substrate comprises a filtration fabric selected from the group consisting of polyester fabric, non-polyester fabric, woven polyester monofilament, polymer and filament, or a combination thereof.
